(19) United States
(12) Patent Application Publication
(10) Pub. No.: US 2021/0369771 A1
(43) Pub. Date: Dec. 2, 2021

(54) PHARMACEUTICAL OR FOOD SUPPLEMENT FORMULATION FOR USE IN TREATING DISORDERS CAUSED BY IRON DEFICIENCY

(71) Applicant: PROSOL S.p.A., Madone (BG) (IT)

(72) Inventors: Daniele Bonvicini, Monza (IT); Nadia Pedretti, Carugate (MI) (IT); Alessandro Sgherbini, Milano (IT)

(73) Assignee: PROSOL S.p.A., Madone (BG) (IT)

(21) Appl. No.: 17/319,297

(22) Filed: May 13, 2021

(30) Foreign Application Priority Data
May 26, 2020 (IT) 102020000012373

(51) Int. Cl. A61K 33/26; A61K 31/708; A61K 31/7076; A61K 31/7068; A61K 31/7072; A61K 31/198; A61K 31/405; A61K 36/064; A61P 3/02

(52) U.S. Cl. CPC A61K 33/26; A61K 31/708; A61K 31/7076; A61K 31/7068; A61P 3/02; A61K 31/198; A61K 31/405; A61K 36/064; A61K 31/7072

(57) ABSTRACT

The present invention relates to a method of treating disorders caused by iron deficiency, comprising administering to a subject in need thereof a therapeutically effective amount of a formulation comprising, as active ingredients, a composition comprising at least 40% of 5′-ribonucleotides by weight of the total weight of the composition, and an inorganic iron salt.

This application claims priority to and the benefit of Italian Application No. 102020000012373 filed on May 26, 2020, the content of which is incorporated by reference in its entirety.

### APPLICATION FIELD

The present invention relates to the pharmaceutical industry field; in particular, the invention relates to a pharmaceutical or food supplement formulation comprising a composition based on 5′-ribonucleotides and an inorganic iron salt, as active ingredients, useful in particular in treating disorders caused by iron deficiency.

### PRIOR ART

It is known that iron is an indispensable element for our body, as its main function concerns the production of hemoglobin, as well as the production of myoglobin, the protein responsible for fixing oxygen in muscle tissues.

This microelement is also involved in the synthesis of collagen and is indispensable in the processes of cellular respiration and in the metabolism of nucleic acids.

Iron is typically absorbed in the duodenum and in the first part of the jejunum (i.e., the central section of the small intestine, preceded by duodenum and followed by ileum in mammals) and, in particular, its absorption is influenced by the chemical form of the iron molecule.

The best iron absorption occurs when ingested foods contain iron in heme form, wherein iron is bound to hemoglobin or myoglobin.

Non-heme iron (in which iron is bound to storage proteins, like ferritin) is usually in a ferric state and must be reduced to a ferrous state and cleaved from the bond with the foods containing it due to the action of gastric secretions.

However, it is also known that iron is hardly absorbed by the body and, consequently, even modest losses, an increased iron demand or a reduced iron absorption can rapidly cause an iron deficiency in the body.

The causes of iron deficiency are quite varied and can be related, more simply, to a diet with a low iron content or to growth in childhood and adolescence, wherein the daily iron demand increases for a correct body growth.

A possible cause of iron deficiency in the body can be also due to pathologies characterized by a prolongated inflammatory state, including inflammatory bowel disease (IBD), Crohn disease, ulcerative colitis, and celiac disease, causing a sensible reduction of the iron amount absorbed at the intestinal level.

Nowadays, the different therapeutic strategies allowing to cope with iron deficiency, in its various levels of severity and supplementation needs, comprise diet modification, oral iron-based food supplements intake, up to injection therapies administration.

With particular reference to iron-based supplements, it is advisable taking them together with vitamin C-rich foods.

In fact, it is known that vitamin C is one of the best molecules to increase the non-heme iron intake at intestinal level.

The commercial product FeRNApyd consists of gastro-resistant capsules comprising iron pidolate (ferrous salt of pidolic acid), nucleotides and vitamin C, wherein the pidolic acid acts as iron organic carrier to tissues, and vitamin C contributes to enhance iron absorption.

Another commercially available product based on vitamin C function to enhance iron absorption at intestinal level is the food supplement “Capifer”, based on micro-incapsulated iron (liposomal iron), nucleotides, vegetable extracts, and a pool of vitamins, including vitamin C, B1, B2 B3, B6, and folic acid (vitamin B9).

It is also commercially available the food supplement “Ferro Difesa 3+”, comprising micro-incapsulated ferric pyrophosphate (ferric pyrophosphate, corn starch, sunflower lecithin and dog rose (Rosa canina L.), vitamin C and guanosine 5′-monophosphate.

The problem underlying the present invention is that of providing an alternative pharmaceutical or food supplement formulation of natural origin, for the treatment of disorders caused by iron deficiency.

### SUMMARY OF THE INVENTION

The present invention solves the aforesaid technical problem by providing a method of treating disorders caused by iron deficiency, comprising administering to a subject in need thereof a therapeutically effective amount of a formulation comprising, as active ingredients, a composition comprising at least 40% of 5′-ribonucleotides by weight of the total weight of the composition, and an inorganic iron salt.

Preferably, the composition comprises 10.4% to 18.2%, more preferably 13% to 15.6% and advantageously 14.3%, of disodium salt heptahydrate of adenosine 5′-monophosphate, 10.4% to 18.2%, more preferably 13% to 15.6% and advantageously 14.3%, of disodium salt heptahydrate of uridine 5′-monophosphate, 9.1% to 18.2%, more preferably 11.7% to 15.6% and advantageously 13%, of disodium salt heptahydrate of cytidine 5′-monophosphate, and 11.7% to 19.5%, more preferably 14.3% to 16.9% and advantageously 15.6%, of disodium salt heptahydrate of guanosine 5′-monophosphate by weight of the total weight of the composition.

Preferably, the composition further comprises 15% to 22%, more preferably 17% to 20% and advantageously 18% of compounds selected from nucleosides and nucleotides different from 5′-ribonucleotides by weight of the total weight of the composition.

Preferably, the composition further comprises 1% to 5%, more preferably 2% to 3%, of a mixture of amino acids by weight of the total weight of the composition.

Preferably, the aforesaid mixture of amino acids comprises methionine, cysteine, threonine, phenylalanine, tryptophan, and lysine.

Preferably, the aforesaid composition is obtained from an extract of a fungal microorganism.

Preferably, the aforesaid fungal microorganism is a yeast belonging to a genus selected from the group comprising Saccharomyces, Kluyveromyces or Candida.

Preferably, the aforesaid inorganic iron salt is selected from the group comprising ferrous sulphate, ferrous carbonate, ferrous phosphate, ferric diphosphate, more preferably ferrous sulphate.

Preferably, the present formulation further comprises a carrier acceptable from the pharmaceutical or food standpoint.

Preferably, the formulation is characterized in that it is in the form of tablets, syrups, capsules, film-coated tablets or sachets of powder or granules.

The Applicant has surprisingly found out the unexpected property of 5′-ribonucleotides, in association with an inorganic iron salt, of promoting a progressive and controlled iron absorption in the intestinal tissue, even if it is known that inorganic iron salts are generally more difficult to be absorbed by the body compared to the respective organic forms or if associated to specific biological carriers.

The Applicant has further surprisingly found out that the mixture of 5′-ribonucleotides and an inorganic iron salt advantageously shows a synergic activity enhancing long term iron absorption, compared to the respective biological activities of the individual components thereof, as shown in the below Examples.

The term “inorganic iron salt”, as used herein, means an inorganic iron salt, suitable for consumption, selected from the group comprising ferrous sulphate, ferrous carbonate, ferrous phosphate, ferric diphosphate.

Advantageously, the solid dosage forms for the administration by oral route comprise, for example, capsules, tablets, powders, granules, and gels. In such solid dosage forms, the active ingredient can be admixed with at least one inert diluent such as, for example, saccharose, lactose or starch. These dosage forms generally also comprise additional substances different from inert diluents, such as, for example, lubricating agents as magnesium stearate.

The pharmaceutical or food supplement preparations for use according to the present invention may be produced by using conventional pharmaceutical techniques, as described in the various pharmacopoeias or handbooks such as, for example, “Remington's Pharmaceutical Sciences Handbook”, Mack Publishing, New York, 18th Ed., 1990.

The average daily dosage of the 5′-ribonucleotides contained in the formulation according to the present invention depends on many factors, such as, for example, disease severity and patient conditions (age, weight, sex): the dose may generally vary from 10 mg to 2000 mg per day, preferably from 300 mg to 1000 mg per day of 5′-ribonucleotides, optionally divided into more administrations.

Even the average daily dosage of the inorganic iron salt contained in the formulation according to the present invention depends on different factors, including age, sex and particular conditions as pregnancy and breastfeeding: the dose may generally vary between 5-30 mg per day, preferably between 10-20 mg of iron per day, optionally divided into more administrations.

### BRIEF DESCRIPTIONS OF FIGURES

FIGS. 1A-1C. FIG. 1A shows a bar graph relating to the cell vitality of Caco-2 cells with varying concentrations (μg/ml) of the digested product of RIBODIET®, obtained as described in the Examples.

FIG. 1B shows a bar graph relating to the cell vitality of Caco-2 cells with varying concentrations (μg/ml) of the combination consisting of the digested product of RIBODIET®, obtained as described in the Examples, and the digested product of iron sulphate heptahydrate (Fe).

FIG. 1C shows a bar graph relating to the cell vitality of Caco-2 cells with varying concentrations (μg/ml) of the digested product of iron sulphate heptahydrate (Fe).

FIG. 2 shows a bar graph relating to the amount of iron (μg) absorbed in the Caco-2 intestinal cells in the presence of the digested product of RIBODIET® (sample A), the combination consisting of the digested product of RIBODIET®, and the digested product of iron sulphate heptahydrate (Fe, sample B), and the digested product of iron sulphate heptahydrate (Fe, sample C), respectively.

### DETAILED DESCRIPTION OF THE INVENTION

For several years, the Applicant has been producing a composition based on 5′-ribonucleotides, marketed under the name RIBODIET® as food supplement, having anti-inflammatory and immunostimulant functions in promoting the health of the intestinal tract.

In view of the beneficial effects found with the use as dietetic supplement of the aforementioned composition, due to its anti-inflammatory and immunizing properties, and the ease of industrial production of RIBODIET® product, the Applicant decided to verify if this product had a positive effect also in treating disorders caused by iron deficiency in the body.

In particular, it has been tested if the product RIBODIET®, based on 5′-ribonucleotides, in combination with an inorganic iron salt, enhanced iron absorption at intestinal level, by a series of in vitro tests.

First, it has been tested if such combination had any cytotoxic effect on the intestinal epithelium. The result is that neither RIBODIET® alone, nor in combination with an inorganic iron salt, in particular iron sulphate, slows the cellular growth at any of the tested concentrations.

On the contrary, it has been highlighted that iron sulphate, alone, exerts a cytotoxic effect on intestinal cells, when tested at high concentrations.

This result means that an excessive iron amount taken in a single dose does not exert a beneficial effect on the intestinal cells responsible for micro-nutrients absorption, but on the contrary produces cytotoxic effect on them, slowing their growth.

In the light of this result, the Applicant investigated if the combination of RIBODIET® and an inorganic iron salt, in particular iron sulphate, had any beneficial effect on the progressive long-term iron absorption at the intestinal level.

As demonstrated in the comparative test reported at the Example 3, the combination of RIBODIET® and iron sulphate has surprisingly determined a progressive iron absorption over a long period of time.

The tested maximum time period was 3 hours, simulating in this way the digestion and the intake of micro-nutrients by the intestinal cells in an adult mammal.

Differently from the aforementioned combination, RIBODIET® tested alone has not exerted any detectable effect on iron absorption in the cells.

Also iron sulphate, tested alone, determined a progressive iron intake by the intestinal cells but, anyway, significantly lower than that determined by the combination of RIBODIET® and iron sulphate.

Consequently, the formulation according to the present invention comprising the product RIBODIET®, based on 5′-ribonucleotides, and iron sulphate, not only promotes intestinal cells growth also at high concentrations (being therefore free of cytotoxic effects), but advantageously exerts also a progressive long-term iron absorption in the intestinal cells, ensuring the intake of this micro-nutrient in the recommended average daily doses.

The method for producing RIBODIET®, sold by the Applicant PROSOL S.p.a., has been disclosed in the Italian patent application N. 102016000112436, and it is briefly summarized below.

The starting raw material is the liquid obtained by the RNA extraction process from yeasts (for example, Kluyveromyces or Saccharomyces).

Such liquid was filtered by microfiltration (membranes with a pore size of 0.45 μm) to separate the suspended particles from the process liquid.

This process liquid has 10% dry substance, which has a hydrolysable RNA content between 60% and 90% (Schmidt & Tannhauser method).

A first dilution is carried out by adding osmotic water, and the pH, if required, is adjusted up to a value of 5.5.

The thus obtained mass is subjected to a first heat treatment step, at a temperature between 90° C. and 100° C. for 20-30 minutes; then, it is cooled by adding water in an amount sufficient to lower the temperature to 70° C.

As the heating step normally causes a drop in pH of 0.2 to 0.5 points, a second pH correction is made by adding 30% NaOH, reporting the value in a range between 5.3 and 5.5.

The thus obtained conditions (70° C. and pH between 5.3 and 5.5) are those considered optimal for the activity of the enzyme necessary to hydrolyze the RNA (ribonuclease).

The enzyme is weighted (0.33% on dry substance) and added to the reactor, after being dissolved in a separated vessel in 10-15 L of osmotic water.

Hydrolysis is carried out for 10 hours at a temperature of 70° C.; in this step, pH decreases due to enzymic activity (decrease of 0.4-0.7 points).

After hydrolysis, the pH is adjusted to 6.30 by adding 30% NaOH. Then, the mass is subjected to a centrifugation step in a clarifying centrifuge and to a subsequent concentration step in a concentrator under vacuum.

Therefore, 1400-1500 L of concentrated liquid having a dry substance of 32-37% are obtained.

The concentrated liquid is then cooled and, finally, pumped to the spray drying system.

The thus obtained formulation in powder form has a 5′-ribonucleotides content from 40% up to 65% (52%-84.5%, considering 5′-ribonucleotides in the heptahydrate disodium salt form), generally between 50-65%.

It also contains nucleosides and other nucleotides different from 5′-ribonucleotides (about 20% w/w) and a mixture of amino acids (about 5% w/w) including methionine, cysteine, threonine, phenylalanine, tryptophan, and lysine.

Further features and advantages of the present invention will become apparent from the following Examples, provided for illustrative and non-limiting purposes.

### EXAMPLES

In order to evaluate the ability of the formulation according to the present invention, comprising 5′-ribonucleotides and an inorganic iron salt, to promote iron absorption in the cells of the intestinal tissue, the below discussed comparative tests have been carried out.

The samples used in the following comparative tests were as follows:

Sample A: food supplement RIBODIET®, produced and marketed by the Applicant, based on 5′-ribonucleotides;

Sample B: combination of RIBODIET® (100 mg) and ferrous sulphate heptahydrate (FeSO₄×7 H₂O) (150 mg, corresponding to 30 mg of Fe);

Sample C: ferrous sulphate heptahydrate (FeSO₄×7 H₂O) sold by the Sigma-Aldrich company (product code: F8633).

### Example 1—Comparative Test on Iron Bioavailability

The above samples were digested in vitro to simulate the physiological process that foods undergo once consumed by the patient and subjected to chemical-physical conditions in the digestive system (oral, gastric, and intestinal phases).

In particular, 100 mg of RIBODIET® (sample A), 150 mg of ferrous sulphate heptahydrate (corresponding to 30 mg of Fe) (sample C) and the aforesaid combination of RIBODIET® and ferrous sulphate heptahydrate (sample B, as defined above), were subjected to a digestion process according to “Versantvoort C H, Oomen A G, Van de Kamp E, Rompelberg C J, Sips A J. Applicability of an in vitro digestion model in assessing the bioaccessibility of mycotoxins from food. Food and Chemicals toxicology, 2005”.

At the end of the digestion process, the iron concentration was determined in the digested products; such concentration was used to determine the overall recovery of the process.

20 mL aliquots of each digested product were subjected to a centrifugation step (2750 g for 5 minutes), and the iron content in the resulting pellets and supernatants was determined, wherein the supernatants corresponded to the iron bioavailable fraction for the cells.

The iron concentration was determined by ICP-MS (inductively coupled plasma mass spectrometry).

The results were reported in the table below.

| | Complete | | Supernatant | | Pellet | |
|---|---|---|---|---|---|---|
| | [Fe] | Fe | [Fe] | Fe | [Fe] | Fe |
| | (μg/mL) | (%) | (μg/mL) | (%) | (μg/mL) | (%) |
| Sample A | n.d. | — | n.d. | — | n.d. | — |
| Sample B | 1870 ± 34 | 118 ± 2 | 503 ± 11 | 27 ± 1 | 1226 ± 131 | 66 ± 7 |
| Sample C | 1701 ± 84 | 108 ± 5 | 623 ± 6 | 37 ± 1 | 1059 ± 69 | 62 ± 4 |

The results show that the iron concentration in the digested product of sample A (RIBODIET®) is below the detection limit of the used method (5 ppm).

The iron amount detected in the supernatant obtained from the digestion of sample C (FeSO₄×7H₂O) is slightly higher than the iron amount detected in the supernatant of sample B (RIBODIET®+FeSO₄×7H₂O).

Further, it was observed that the pellets of the digested products of samples C and B show a similar iron content.

From the above, it was therefore observed that, following the digestion in the tested samples, the supernatants of the digested samples of ferrous sulphate and of the combination of ferrous sulphate and RIBODIET® substantially show the same concentration of iron bioavailable for the intestinal cells.

### Example 2—Comparative Test on Intestinal Epithelium Vitality in the Presence of the Digested Products of Samples A, B and C of the Example 1

To evaluate the impact of the samples A, B and C, as defined in the Example 1, on cell vitality of intestinal epithelium, an in vitro model based on Caco-2 intestinal cells (ATCC, HBT-37™) derived from human adenocarcinoma and cultured as functional monolayers in Transwell® inserts, was used.

These particular inserts consist of an apical compartment, on which the monolayers of Caco-2 cells are deposited, and a basolateral compartment, wherein the apical and basolateral compartments are separated from each other by a microporous membrane.

To perform the test, a toxicological analysis using a dose-response curve was carried out. The bio-accessible fractions (supernatants) of samples A, B and C were diluted in a simulated intestinal fluid (“Simulated Intestinal Fluid” according to the U.S. Pharmacopeia USP 26, described also in Stippler et al., “Dissolution Technologies” 11(2): 6-10, 2004) according to the following progression: 100% (undiluted), 50%, 25%, 17%, 13%, 10%, 8%, 7%, 5% e 0% (simulated intestinal fluid). In the apical compartment of Transwell® inserts, in contact with Caco-2 cell layers, 1 mL of the different dilutions of the obtained bio-accessible fractions was added. In the basolateral compartment of the inserts, 1.5 mL of HBSS buffer (“Hank's Balanced Salt Solution) were added.

The negative control consisted of the simulated intestinal fluid.

After 3 hours incubation, Caco-2 cells vitality was evaluated by MTS assay, based on the reduction of the tetrazole compound MTS (3-(4,5-dimethylthiazole-2-yl)-5-(3-carboxymethoxyphenyl)-2-(4-sulfophenyl)2H-tetrazolium), by mitochondrial dehydrogenases active in living cells, in the colored product formazan, whose concentration can be quantitatively determined by measuring the absorbance of the sample at 490 nm.

Using the MTS assay, the cell vitality is therefore directly proportional to the absorbance measured in the sample.

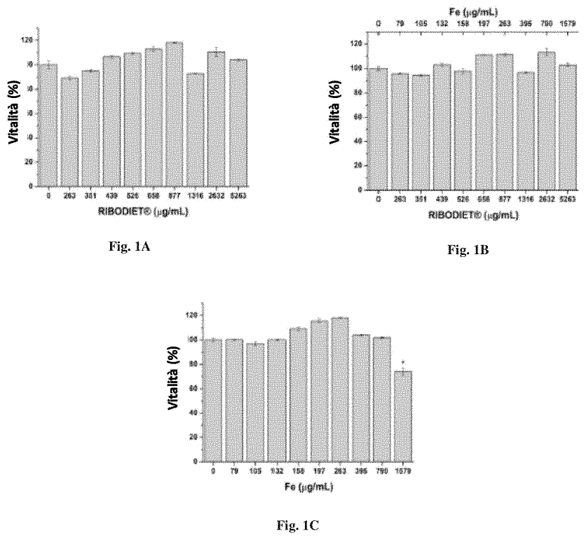

As shown by the results of FIGS. 1A-1C, no cytotoxic effects of sample A (RIBODIET®) and sample B (RIBODIET®+FeSO₄×7H₂O) were detected at any of the tested concentrations.

On the contrary, this test highlighted that, at high concentrations, iron sulphate has a cytotoxic effect on the intestinal cells that, in fact, show a lowered cell vitality when exposed to high concentrations of inorganic iron.

### Example 3—Comparative Test on Iron Absorption Over Time of the Digested Products of Samples a, B and C of the Example 1

To perform this test, the supernatants of the digested samples A, B and C obtained according to the Example 1 were used.

The same sample preparation methodology according to the Example 2 was applied, using Caco-2 cells disposed in Transwell® inserts; in particular, in this test, 10 μL of fetal bovine serum (FBS) were added.

The test was performed according to the following incubation times: 1 hour and 3 hours.

At the end, the Caco-2 cell monolayers were harvested for iron content determination according to the following procedure.

At the end of exposition (1 and 3 hours), the Caco-2 cell monolayer was washed with HBSS, detached from the microporous membrane by trypsinization, centrifuged, and the thus obtained pellet was washed 2 times with PBS.

After removing the supernatant obtained from the last centrifugation, the pellet, consisting of Caco-2 cells forming the monolayer, was dehydrated by vacuum dryer, and suitably processed for the analysis of Fe content in ICP-MS.

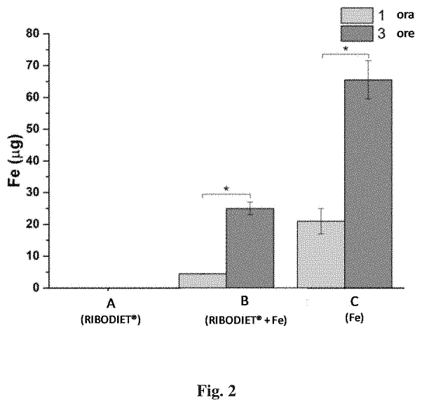

The obtained results are reported in the Table below and in FIG. 2:

| | Intracellular | Intracellular |
|---|---|---|
| | iron (μg) (1 h | iron (μg) (3 h |
| | incubation) | incubation) |
| Sample A | 0 | 0 |
| (RIBODIET ®) | | |
| Sample B | 4.5 | 25.0 |
| (RIBODIET ® + | | |
| FeSO₄ × 7 H₂O) | | |
| Sample C | 21.0 | 65.5 |
| (FeSO₄ × 7 H₂O) | | |

The results show that, in the time elapsed between 1 hour and 3 hours, sample B determined an unexpected increase of iron absorption in the intestinal cells equal to 6×.

Sample C, in the same period of time, determined an increase of iron absorption in the intestinal cells equal to 3×.

Sample A, instead, did not determined any iron absorption in the intestinal cells.

In the light of these results, it is apparent that the combination of RIBODIET® and the aforesaid inorganic iron salt promotes a progressive and controlled iron absorption over time by the intestinal cells, being free from any cytotoxic effect on intestinal cells even at high concentrations.

The use of this combination for treating disorders related to iron deficiency is therefore particularly advantageous.

1. A method of treating disorders caused by iron deficiency, comprising administering to a subject in need thereof a therapeutically effective amount of a formulation comprising, as active ingredients, a composition comprising at least 40% of 5′-ribonucleotides by weight of the total weight of the composition, and an inorganic iron salt.

2. The method according to claim 1, wherein said composition comprises 10.4% to 18.2% of disodium salt heptahydrate of adenosine 5′-monophosphate, 10.4% to 18.2% of disodium salt heptahydrate of uridine 5′-monophosphate, 9.1% to 18.2% of disodium salt heptahydrate of cytidine 5′-monophosphate, and 11.7% to 19.5% of disodium salt heptahydrate of guanosine 5′-monophosphate by weight of the total weight of the composition.

3. The method according to claim 1, wherein said composition further comprises 15% to 22% of compounds selected from nucleosides and nucleotides different from 5′-ribonucleotides by weight of the total weight of the composition.

4. The method according to claim 1, wherein said composition further comprises 1% to 5% of a mixture of amino acids by weight of the total weight of the composition.

5. The method according to claim 4, wherein said mixture of amino acids comprises methionine, cysteine, threonine, phenylalanine, tryptophan, and lysine.

6. The method according to claim 1, wherein said composition is obtained from an extract of a fungal microorganism.

7. The method according to claim 6, wherein said fungal microorganism is a yeast belonging to a genus selected from the group consisting of Saccharomyces, Kluyveromyces or Candida.

8. The method according to claim 1, wherein said inorganic iron salt is selected from the group consisting of ferrous sulphate, ferrous carbonate, ferrous phosphate, and ferric diphosphate.

9. The method according to claim 1, wherein said formulation further comprises a carrier acceptable from the pharmaceutical or food standpoint.

10. The method according to claim 1, characterized in that said formulation is in the form of tablets, syrups, capsules, film-coated tablets or sachets of powder or granules.

11. The method according to claim 2, wherein said composition comprises 13% to 15.6% of disodium salt heptahydrate of adenosine 5′-monophosphate, 13% to 15.6% of disodium salt heptahydrate of uridine 5′-monophosphate, 11.7% to 15.6% of disodium salt heptahydrate of cytidine 5′-monophosphate, and 14.3% to 16.9% of disodium salt heptahydrate of guanosine 5′-monophosphate by weight of the total weight of the composition.
